(19) United States
(12) Patent Application Publication
(10) Pub. No.: US 2025/0115636 A1
(43) Pub. Date: Apr. 10, 2025

(54) PEPTIDOMIMETIC INHIBITORS OF PROTEIN N-TERMINAL METHYLTRANSFERASE 1, COMPOSITION, AND METHOD OF USE

(71) Applicant: Purdue Research Foundation, West Lafayette, IN (US)

(72) Inventors: Rong Huang, West Lafayette, IN (US); Dongxing Chen, West Lafayette, IN (US); Guangping Dong, West Lafayette, IN (US)

(73) Assignee: Purdue Research Foundation, West Lafayette, IN (US)

(21) Appl. No.: 18/729,426

(22) PCT Filed: Dec. 2, 2022
(86) PCT No.: PCT/US22/51697

§ 371 Date: Jul. 16, 2024

Related U.S. Application Data
(60) Provisional application No. 63/306,167, filed on Feb. 3, 2022.

(51) Int. Cl. C07K 5/078; A61K 38/00

(52) U.S. Cl. CPC C07K 5/06139; A61K 38/00

(57) ABSTRACT

Peptidomimetic inhibitors of protein N-terminal methyltransferase 1, such as a compound with the following formula: a composition comprising same, and a method of use.

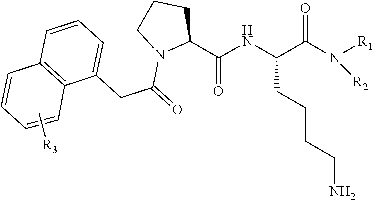

### CROSS REFERENCE TO RELATED APPLICATIONS

This application claims priority to U.S. provisional patent application No. 63/306,167, which was filed Feb. 3, 2022, and which is hereby incorporated by reference in its entirety.

### GOVERNMENT RIGHTS

This invention was made with government support under GM117275 awarded by the National Institutes of Health (NIH). The government has certain rights in the invention.

### TECHNICAL FIELD

The present disclosure relates to peptidomimetic inhibitors of protein N-terminal methyltransferase 1, a composition comprising same, and a method of use.

### BACKGROUND

This section introduces aspects that may help facilitate a better understanding of the disclosure. Accordingly, these statements are to be read in this light and are not to be understood as admissions about what is or is not prior art.

Protein α-N-terminal methyltransferases (NTMTs/NRMTs) catalyze the addition of 1-3 methyl group(s) to the protein α-N-terminus from the cofactor S-adenosyl-L-methionine (SAM). Protein N-terminal methyltransferases 1/2 (NTMT1/2, METTL11A/B) are the two reported members of this family that recognize a unique N-terminal X-P-K/R motif (in which X represents any amino acid other than D/E).^{1, 2} To date, the regulator of chromosome condensation 1 (RCC1), the tumor suppressor retinoblastoma 1 (RB1), oncoprotein SET, centromere protein A/B (CENP-A/B), damaged DNA-binding protein 2 (DDB2), poly(ADP-ribose) polymerase 3 (PARP3), Obg-like ATPase 1 (OLA1), and MORF-related gene 15 (MRG15) have been validated as physiological substrates for NTMT1.³⁻⁷ Though protein α-N-terminal methylation is evolutionarily conserved across different species, the knowledge of its functions remains scant.⁸ Unlike aliphatic side-chain amines (pKa: 10.5), full methylation of the protein α-N-terminal amine (pKa: 6˜8) alters both the hydrophobicity and the charge state.⁹ The resulting positive charge at the protein α-N-terminus strengthens the interactions of a protein with its respective binding partner as exemplified in RCC1 and CENP-A.^{1, 3, 10} α-N-terminal methylation of DDB2 facilitates its nuclear localization to cyclobutane pyrimidine dimer (CPD) foci.⁴ In addition, NTMT1 has been implicated in regulating mitosis, DNA damage repair, stem cell maintenance, and cervical cancer cell proliferation and migration.^{1, 11-13} Thus, cell-potent inhibitors are potentially valuable tools to study the functions and roles of NTMT1/2.

In view of the above, it is an object of the present disclosure to provide peptidomimetic inhibitors of NTMT1. This and other objects and advantages, as well as inventive features, will be apparent from the detailed description provided herein.

### SUMMARY

Provided is a compound of formula (I):

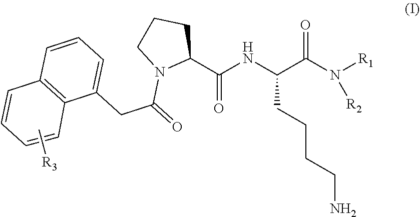

wherein:
R₁ is hydrogen or an alkyl;
R₂ is an alkyl, an alkenyl, an alkynyl, an arylalkyl, a cycloalkyl, a heterocyclyl, an aryl, a heteroaryl, an alkylaryl, or a heteroarylalkyl; and
R₃ represents seven substituents independently selected from hydrogen, an alkyl, an alkenyl, an alkynyl, an acyl, an amino, a cyano, a halo, hydroxy, a arylalkyl, a cycloalkyl, a heterocyclyl, an aryl, a heteroaryl, an alkylaryl, and a heteroarylalkyl;
or a pharmaceutically acceptable salt, hydrate, or solvate thereof. Regarding the compound, (i) R₁ can be optionally substituted, (ii) R₂ can be optionally substituted, (iii) one or more of the R₃ substituents can be optionally substituted, or (iv) two or more of (i)-(iii). In an embodiment, R₁ can be hydrogen, and R₂ can be an arylalkyl, in which the aryl can be substituted with a halo, such as Br, or an aryl. In an embodiment, R₁ can be methyl, and R₂ can be an arylalkyl, in which the aryl can be substituted with a halo, such as Br, or an aryl. In an embodiment, R₁ can be hydrogen, and R₂ can be a cycloalkyl. In another embodiment, R₁ can be methyl, and R₂ can be a cycloalkyl. The compound can be:

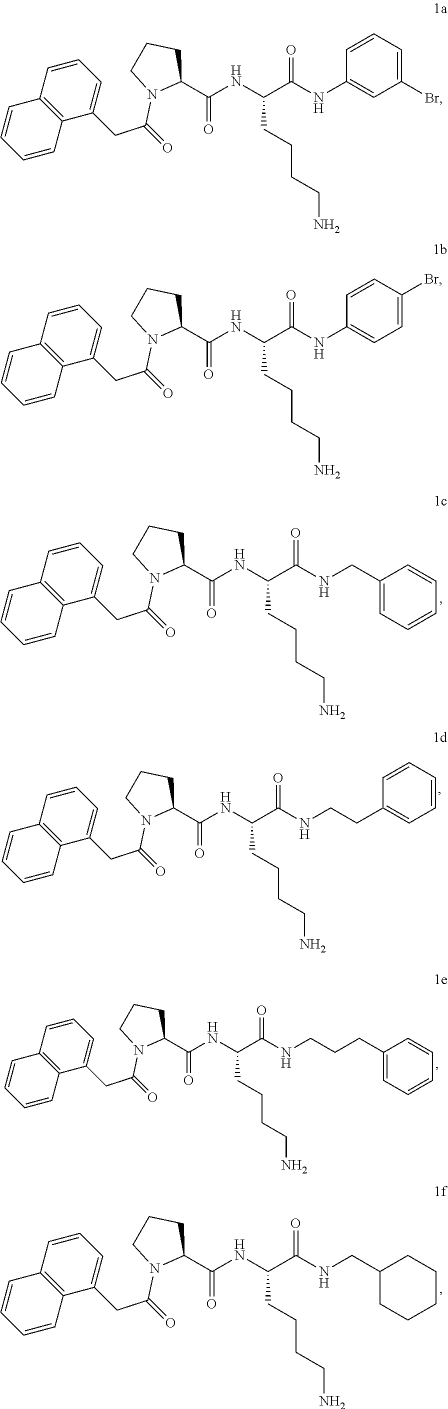 [SMILES: NCCCC[C@H](NC(=O)[C@@H]1CCCN1C(=O)Cc1cccc2ccccc12)C(=O)Nc1cccc(Br)c1 ; NCCCC[C@H](NC(=O)[C@@H]1CCCN1C(=O)Cc1cccc2ccccc12)C(=O)Nc1ccc(Br)cc1 ; NCCCC[C@H](NC(=O)[C@@H]1CCCN1C(=O)Cc1cccc2ccccc12)C(=O)NCc1ccccc1 ; NCCCC[C@H](NC(=O)[C@@H]1CCCN1C(=O)Cc1cccc2ccccc12)C(=O)NCCc1ccccc1 ; NCCCC[C@H](NC(=O)[C@@H]1CCCN1C(=O)Cc1cccc2ccccc12)C(=O)NCCCc1ccccc1 ; NCCCC[C@H](NC(=O)[C@@H]1CCCN1C(=O)Cc1cccc2ccccc12)C(=O)NCC1CCCCC1]

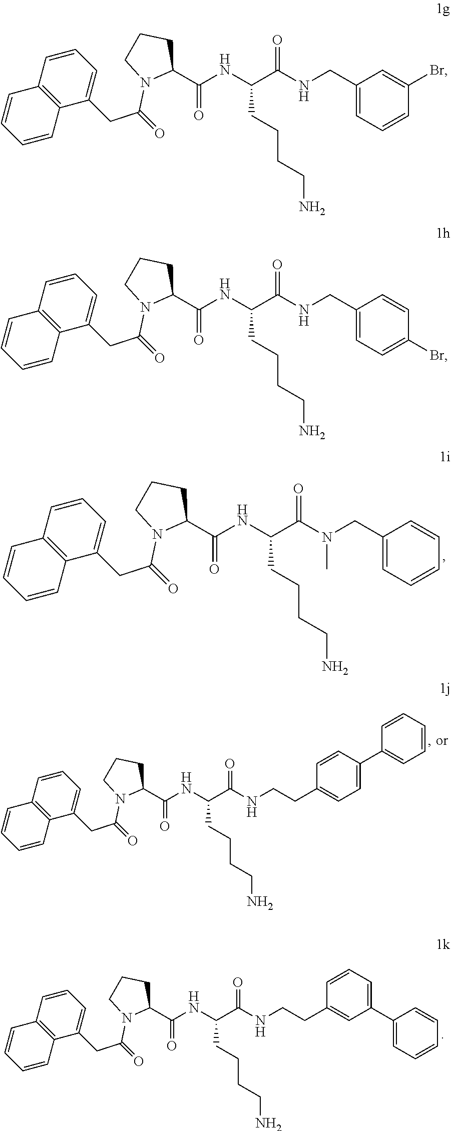 [SMILES: NCCCC[C@H](NC(=O)[C@@H]1CCCN1C(=O)Cc1cccc2ccccc12)C(=O)NCc1cccc(Br)c1 ; NCCCC[C@H](NC(=O)[C@@H]1CCCN1C(=O)Cc1cccc2ccccc12)C(=O)NCc1ccc(Br)cc1 ; CN(Cc1ccccc1)C(=O)[C@H](CCCCN)NC(=O)[C@@H]1CCCN1C(=O)Cc1cccc2ccccc12 ; NCCCC[C@H](NC(=O)[C@@H]1CCCN1C(=O)Cc1cccc2ccccc12)C(=O)NCCc1ccc(-c2ccccc2)cc1 ; NCCCC[C@H](NC(=O)[C@@H]1CCCN1C(=O)Cc1cccc2ccccc12)C(=O)NCCc1cccc(-c2ccccc2)c1]

The compound can, and desirably does, inhibit NTMT1.

In view of the above, also provided is a pharmaceutical composition comprising an above-described compound and a pharmaceutically acceptable carrier, diluent, excipient or a combination thereof. The compound can, and desirably does, inhibit NTMT1.

In further view of the above, also provided is a method of inhibiting NTMT1 in a subject in need thereof. The method comprises administering an effective amount of (i) an above-described compound, which inhibits NTMT1, or (ii) a pharmaceutical composition comprising an above-described compound, which inhibits NTMT1, and a pharmaceutically acceptable carrier, diluent, excipient, or a combination thereof. The patient can have cancer, such as cervical, prostate, lung, breast, colorectal, or pancreatic cancer, melanoma, or neuroblastoma.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures. The drawings are for illustration and not necessarily to scale.

FIG. 1. Optimization strategy of cell-potent peptidomimetic N-terminal methyltransferase 1 (NTMT1) inhibitors.

FIGS. 2A-2D. Docking studies of the peptidomimetic inhibitors through Glide. Linear regression model of IC₅₀ values and docking scores of the inhibitors (FIG. 2A). Representative docking models of GD556 (FIG. 2B), GD590 (FIG. 2C), and GD591 (FIG. 2D), respectively. The NTMT1 is shown as white ribbon. The peptidomimetic inhibitors are shown as black sticks, and the interacting residues are shown in the white sticks. The H-bonds are shown as dotted lines.

FIGS. 3A-3B. Cytotoxicity studies of the peptidomimetic inhibitors via alamarBlue assay (n=3). The effects of all the peptidomimetic inhibitors on growth in normal HCT116 cells (FIG. 3A). The effects of 1a on growth in both normal and NTMT1 knockout (KO) HCT116 cells (FIG. 3B).

FIGS. 4A-4C. Cellular N-terminal methylation assay of GD562 (1d), GD564 (1f), and GD573 (1e) in HCT116 cells. Representative Western blot results of the effects of GD562 (0-300 μM) on the cellular methylation level (n=2) (FIG. 4A). Representative Western blot results of the effects of GD564 (0-100 μM) on the cellular methylation level (n=2) (FIG. 4B). Representative Western blot results of the effects of GD573 (0-300 μM) on the cellular methylation level (n=2) (FIG. 4C). Image quantification was done using ImageJ software (NIH). All bands were compared to the respective untreated control, which was set at 1.0.

FIG. 5. In-house selectivity studies of GD562. The compound was tested at 100, 30, and 10 μM (n=2). Both SAM and substrate are at their Kₘ values in the studies.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Those skilled in the art will recognize that numerous modifications can be made to the specific implementations described above. The implementations should not be limited to the particular limitations described. Other implementations may be possible.

Guided by the co-crystal structures of N-terminal methyltransferase 1 (NTMT1) in complex with the previously reported peptidomimetic inhibitor DC113, a series of new peptidomimetic inhibitors was designed and synthesized. Through a focused optimization of DC113, a new cell-potent peptidomimetic inhibitor was discovered. GD562 (IC₅₀=0.93±0.04 μM) exhibited improved inhibition on cellular N-terminal methylation levels of both the regulator of chromosome condensation 1 and oncoprotein SET with an IC₅₀ value of ˜50 μM in human colorectal cancer HCT116 cells. Notably, GD562's inhibition of the SET protein was over six-fold greater than that of the previously reported cell-potent inhibitor DC541. Furthermore, GD562 also exhibited over 100-fold selectivity for NTMT1 as compared to several other methyltransferases.

In view of the above, provided is a compound of formula (I):

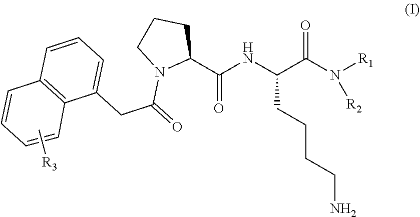

wherein:
R₁ is hydrogen or an alkyl;
R₂ is an alkyl, an alkenyl, an alkynyl, an arylalkyl, a cycloalkyl, a heterocyclyl, an aryl, a heteroaryl, an alkylaryl, or a heteroarylalkyl; and
R₃ represents seven substituents independently selected from hydrogen, an alkyl, an alkenyl, an alkynyl, an acyl, an amino, a cyano, a halo, hydroxy, a arylalkyl, a cycloalkyl, a heterocyclyl, an aryl, a heteroaryl, an alkylaryl, and a heteroarylalkyl;
or a pharmaceutically acceptable salt, hydrate, or solvate thereof. Regarding the compound, (i) R₁ can be optionally substituted, (ii) R₂ can be optionally substituted, (iii) one or more of the R₃ substituents can be substituted, or (iv) two or more of (i)-(iii). In an embodiment, R₁ can be hydrogen, and R₂ can be an arylalkyl, in which the aryl can be substituted with a halo, such as Br, or an aryl. In an embodiment, R₁ can be methyl, and R₂ can be an arylalkyl, in which the aryl can be substituted with a halo, such as Br, or an aryl. In an embodiment, R₁ can be hydrogen, and R₂ can be a cycloalkyl. In another embodiment, R₁ can be methyl, and R₂ can be a cycloalkyl. The compound can be:

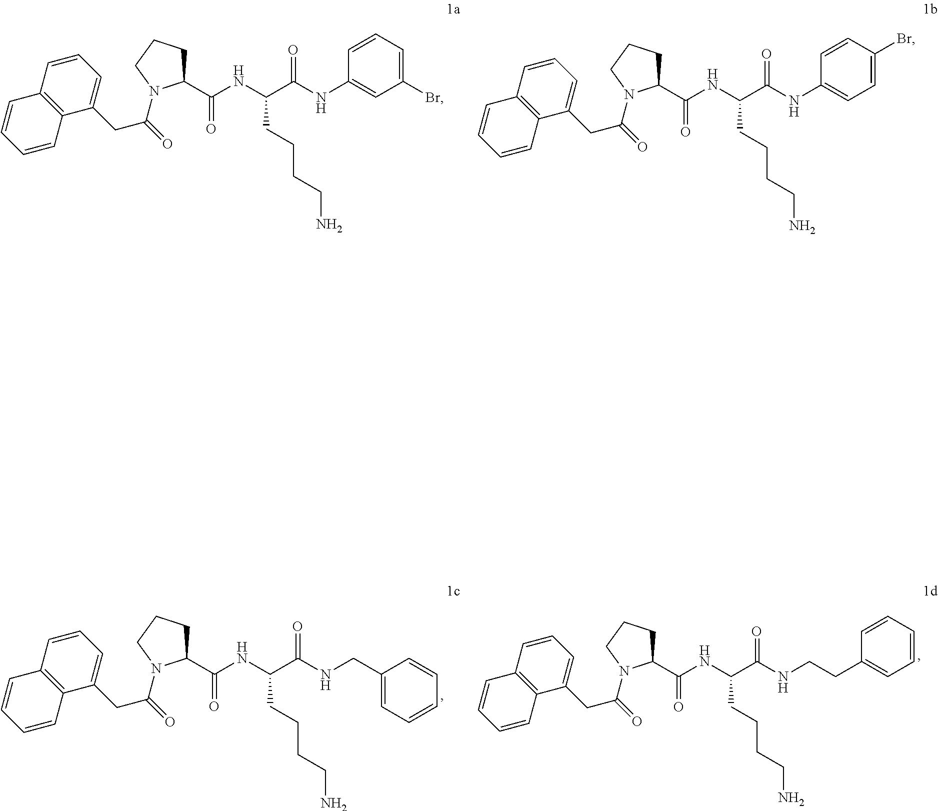

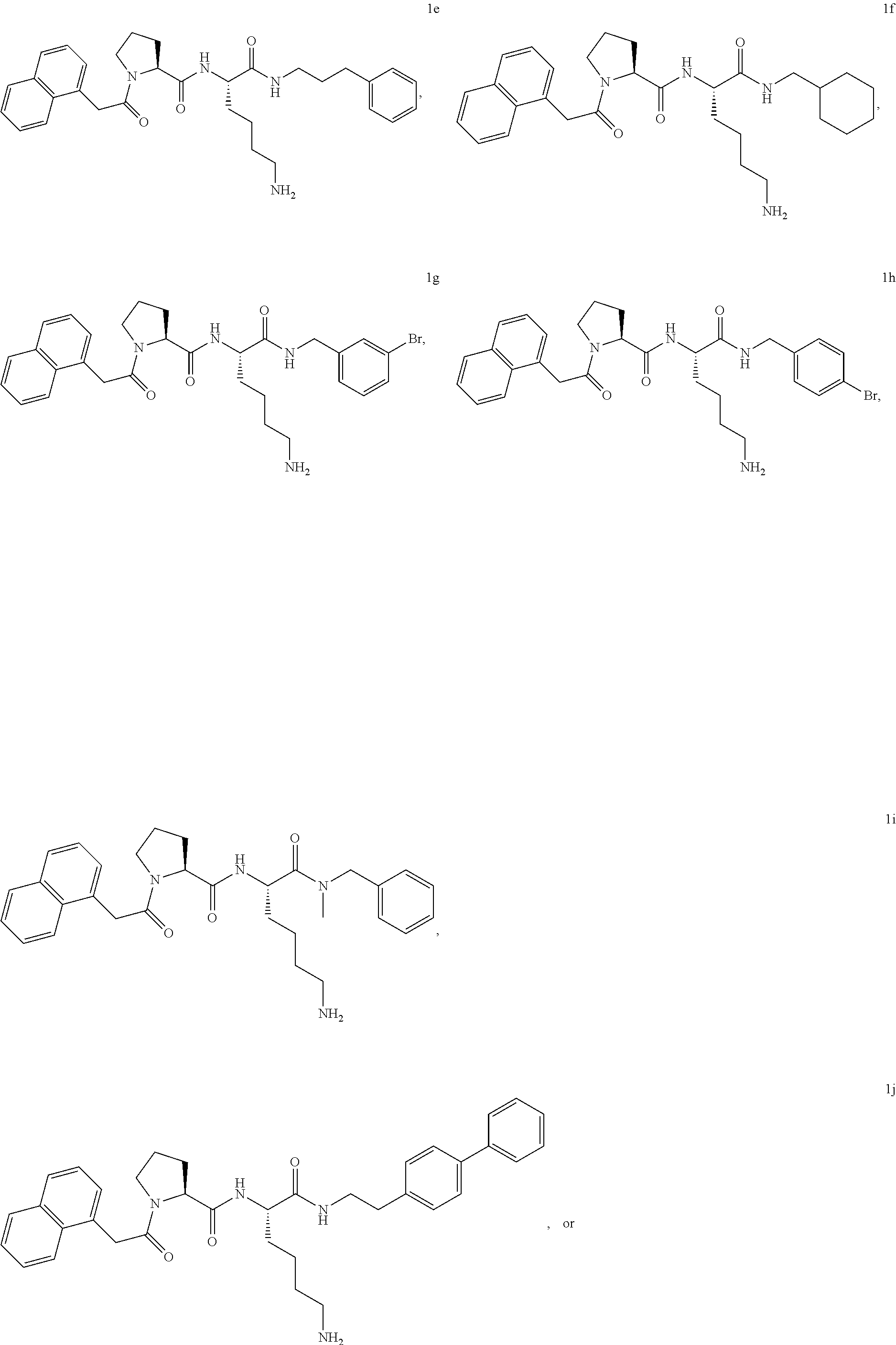

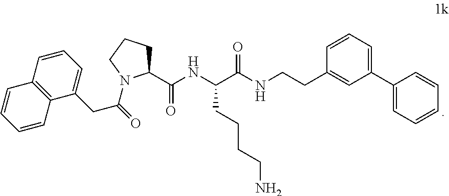 [SMILES: NCCCC[C@H](NC(=O)[C@@H]1CCCN1C(=O)Cc1cccc2ccccc12)C(=O)NCCc1cccc(-c2ccccc2)c1]

The compound can, and desirably does, inhibit protein NTMT1.

The compounds can be synthesized in accordance with methods known in the art. Other methods are exemplified herein.

In each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The compounds described herein may contain one or more chiral centers, or may otherwise be capable of existing as multiple stereoisomers. It is to be understood that in one embodiment, the compounds described herein, as well as compositions comprising the compounds and methods of using the compounds/compositions, are not limited to any particular stereochemical requirement and may be optically pure or any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configurations at one or more other chiral centers.

Similarly, the compounds described herein may be include geometric centers, such as cis or trans, e.g., E and Z, double bonds. It is to be understood that the compounds described herein, as well as compositions comprising the compounds and methods of using the compounds/compositions, are not limited to any particular geometric isomer requirement and may be pure or any of a variety of geometric isomer mixtures. It is also to be understood that such mixtures of geometric isomers may include a single configuration at one or more double bond(s), while including mixtures of geometry at one or more other double bonds.

The term “positional isomer” refers to structural isomers around a central ring, such as ortho-, meta-, and para-isomers around a benzene ring. Further, it is understood that replacement of one or more hydrogen atoms with deuterium can significantly lower the rate of metabolism of a drug and, therefore, increase its half-life.

The term “organic group” as used herein refers to, but is not limited to, any carbon-containing functional group. For example, an organic group can be an oxygen-containing group, such as an alkoxy group, an aryloxy group, an aralkyloxy group, an oxo (carbonyl) group, and a carboxyl group (including a carboxylic acid, a carboxylate, and a carboxylate ester), a sulfur-containing group (including an alkyl sulfide and an aryl sulfide), or another heteroatom-containing group.

The term “substituted” as used herein refers to an organic group as defined herein or molecule in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as a hydroxyl group, an alkoxy group, an aryloxy group, an aralkyloxy group, an oxo (carbonyl) group, and a carboxyl group (including a carboxylic acid, a carboxylate, and a carboxylate ester); a sulfur atom in groups such as a thiol group, an alkyl sulfide group, an aryl sulfide group, a sulfoxide group, a sulfone group, a sulfonyl group, and a sulfonamide group; a nitrogen atom in groups such as an amine, a hydroxylamine, a nitrile, a nitro group, an N-oxide, a hydrazide, an azide, and an enamine; and other heteroatoms in various other groups.

The term “alkyl” refers to substituted and unsubstituted, straight-chain and branched alkyl groups and cycloalkyl groups having from 1 to 40 carbon atoms (C₁-C₄₀), 1 to 20 carbon atoms (C₁-C₂₀), 1 to 12 carbon atoms (C₁-C₁₂), 1 to 8 carbon atoms (C₁-C₈), or, in some embodiments, 1 to 6 carbon atoms (C₁-C₆). Examples of straight-chain alkyl groups include those with 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. The term “alkyl” encompasses n-alkyl, isoalkyl, and anteisoalkyl groups as well as other branched forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” refers to substituted and unsubstituted, straight-chain and branched divalent alkenyl and cycloalkenyl groups having from 2 to 20 carbon atoms (C₂-C₂₀), 2 to 12 carbon atoms (C₂-C₁₂), 2 to 8 carbon atoms (C₂-C₈) or, in some embodiments, 2 to 4 carbon atoms (C₂-C₄) and at least one carbon-carbon double bond. Examples of straight-chain alkenyl groups include those with 2 to 8 carbon atoms, such as —CH═CH—, —CH═CHCH₂—, and the like. Examples of branched alkenyl groups include, but are not limited to, —CH═C(CH₃)— and the like.

The term “alkylene” as used herein refers to substituted and unsubstituted, straight-chain and branched divalent alkylene groups and cycloalkylene groups having from 1 to 40 carbon atoms (C₁-C₄₀), 1 to 20 carbon atoms (C₁-C₂₀), 1 to 12 carbon atoms (C₁-C₁₂), 1 to 8 carbon atoms (C₁-C₈) or, in some embodiments, 1 to 4 carbon atoms (C₁-C₄), 1 to 5 carbon atoms (C₁-C₅), 2 to 5 carbon atoms (C₂-C₅) or 3 to 4 carbon atoms (C₃-C₄). Examples of straight-chain alkylene groups include those with 1 to 8 carbon atoms such as methylene (—CH₂—), ethylene (—CH₂CH₂—), n-propylene (—CH₂CH₂CH₂—), n-butylene (—CH₂ (CH₂)₂CH₂—) and the like. Examples of branched alkylene groups include, but are not limited to, isopropylidene (CH₂CH(CH₃)) and the like. Examples of cycloalkylene groups include, but are not limited to, cyclopropylidene, cyclobutylidene, cyclopentylidene and the like.

The term “alkynyl” refers to hydrocarbyl moieties of the scope of alkenyl but having one or more triple bonds.

The term “hydroxyalkyl” refers to alkyl groups substituted with at least one hydroxyl (—OH) group.

The term “cycloalkyl” refers to substituted and unsubstituted, cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to 8-12 ring members, whereas in other embodiments the number of ring carbon atoms ranges from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. The term “cycloalkyl” may be used to refer to the substituent when it is attached to the remainder of the compound by an atom of the cyclo portion of the substituent. The term “alkylcyclyl” may be used to refer to the substituent when it is attached to the remainder of the compound by an atom of the alkyl portion of the substituent. The terms “cycloalkyl” and “alkylcyclyl” may be used interchangeably herein. Put another way, “cycloalkyl” may be used to refer to the substituent when it is attached to the remainder of the compound by an atom of the cyclo portion of the substituent or by an atom of the alkyl portion of the substituent.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclylalkyl, heteroaryl, heteroarylalkyl group or the like. In the special case where the carbonyl carbon atom is bonded to a hydrogen, the group is a “formyl” group, i.e., an acyl group. An acyl group can include 0 to about 12-40, 6-10, 1-5 or 2-5 additional carbon atoms bonded to the carbonyl group. An acryloyl group is an example of an acyl group. An acyl group can also include heteroatoms. A nicotinoyl group (pyridyl-3-carbonyl) is an example of an acyl group. Other examples include acetyl, benzoyl, phenylacetyl, pyridylacetyl, cinnamoyl, acryloyl groups, and the like. When the group containing the carbon atom that is bonded to the carbonyl carbon atom contains a halogen, the group is termed a “haloacyl” group. An example of a haloacyl group is a trifluoroacetyl group.

The term “heterocyclylcarbonyl” is an example of an acyl group that is bonded to a substituted or unsubstituted heterocyclyl group. An example of a heterocyclylcarbonyl group is a prolyl group, wherein the prolyl group can be a D- or an L-prolyl group.

The term “aryl” refers to substituted and unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6 to 14 carbon atoms (C₆-C₁₄) or from 6 to 10 carbon atoms (C₆-C₁₀) in the ring portions of the groups. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups. An aryl can be mono-, bi-, or polycyclic.

The term “heteroaryl” refers to substituted and unsubstituted aromatic 3-12 membered ring structures, 5-12 membered ring structures, or 5-10 membered ring structures, in which a ring includes 1-4 heteroatoms. Heteroaryl groups include, for example, pyrrole, furan, thiophene, imidazole, oxazole, thiazole, triazole, pyrazole, pyridine, pyrazine, pyridazine, pyrimidine, and the like. A heteroaryl can be mono-, bi-, or polycyclic.

The terms “aralkyl” and “arylalkyl” refer to alkyl groups in which a hydrogen or carbon bond of an alkyl group is replaced with a bond to an aryl group. Representative aralkyl groups include, but are not limited to, benzyl and phenylethyl groups and fused (cycloalkylaryl)alkyl groups, such as 4-ethyl-indanyl. The terms “alkylaryl” and “arylalkyl” refer to an aryl substituted with an alkyl group. The term “alkylaryl” may be used to refer to the substituent when it is attached to the remainder of the compound by an atom on the alkyl portion of the substituent. The term “arylalkyl” may be used to refer to the substituent when it is attached to the remainder of the compound by an atom of the aryl portion of the substituent. The terms “alkylaryl” and “arylalkyl” may be used interchangeably herein. Put another way, “arylalkyl” may be used to refer to the substituent when it is attached to the remainder of the compound by an atom of the aryl portion of the substituent or by an atom of the alkyl portion of the substituent. The terms “aralkenyl” and “arylalkenyl” refer to alkenyl groups in which a hydrogen or carbon bond of an alkyl group is replaced with a bond to an aryl group.

The term “heterocyclyl” refers to substituted and unsubstituted aromatic and non-aromatic ring groups containing 3 or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to 20 ring members, whereas other such groups have 3 to 15 ring members. In some embodiments, heterocyclyl groups have 3 to 8 carbon atoms (C₃-C₈), 3 to 6 carbon atoms (C₃-C₆), or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl can also include one or more double bonds. A “heteroaryl” group is an embodiment of a heterocyclyl group. The phrase “heterocyclyl group” includes fused ring species, including those that include fused aromatic and non-aromatic groups. Representative heterocyclyl groups include, but are not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heteroarylalkyl” refers to alkyl groups in which a hydrogen or carbon bond of an alkyl group is replaced with a bond to a heteroaryl group.

The term “amine” as used herein refers to primary, secondary, and tertiary amines. Amines include, but are not limited to. R—NH₂, for example, alkylamines, arylamines, and alkylarylamines; R₂NH, wherein each R is independently selected, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions.

The term “amino group” refers to a substituent of the form —NH₂, —NHR, —NR₂, and —NR₃⁺, wherein each R is independently selected and includes protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino, a dialkylamino, and a trialkylamino group.

The term “cyano” means-CN.

The terms “halo,” “halogen,” and “halide” group, by themselves or as part of another substituent, mean, unless otherwise stated, a fluorine, chlorine, bromine, or iodine atom.

The term “hydroxy” means-OH.

The term “haloalkyl” group includes mono-halo alkyl groups, poly-halo alkyl groups, in which all halo atoms can be the same or different, and per-halo alkyl groups, in which all hydrogen atoms are replaced by halogen atoms, such as fluoro. Examples of haloalkyl include trifluoromethyl, 1,1-dichloroethyl, perfluorobutyl, —CF(CH₃)₂ and the like.

The compounds can contain one or more asymmetric centers and thus give rise to enantiomers, diastereomers, and other stereoisomeric forms that are defined, in terms of absolute stereochemistry, as (R)- or(S)—. Unless stated otherwise, it is intended that all stereoisomeric forms of the compounds are contemplated. When the compounds described herein contain alkene double bonds, and unless specified otherwise, it is intended that this disclosure includes both E and Z “geometric isomers” (e.g., cis or trans). Likewise, all possible isomers, as well as their racemic and optically pure forms, and all tautomeric forms are also intended to be included. The term “positional isomer” refers to structural isomers around a central ring, such as ortho-, meta-, and para-isomers around a benzene ring. Further, it is understood that replacement of one or more hydrogen atoms with deuterium can significantly lower the rate of metabolism of a drug and, therefore, increase its half-life.

As used herein, the terms “salts” and “pharmaceutically acceptable salts” refer to derivatives of the compounds wherein the parent compound is modified by making an acid or base salt thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids, such as hydrochloric, hydrobromic, sulfuric, sulfamic, phosphoric, and nitric, and the salts prepared from organic acids, such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, and acetonitrile are preferred. Lists of suitable salts are found in Remington's Pharmaceutical Sciences, 17th ed., Mack Publishing Company, Easton, Pa., 1985.

The term “solvate” means a compound, or a salt thereof, that further includes a stoichiometric or non-stoichiometric amount of solvent bound by non-covalent intermolecular forces. When the solvent is water, the solvate is a “hydrate”.

The term “prodrug” means a derivative of a compound that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as amides, esters, carbamates, carbonates, ureides, and phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

In view of the above, also provided is a pharmaceutical composition comprising an above-described compound and a pharmaceutically acceptable carrier, diluent, excipient or a combination thereof. The compound can, and desirably does, inhibit NTMT1.

A “pharmaceutical composition” refers to a chemical or biological composition suitable for administration to a subject (e.g., mammal). Such compositions may be specifically formulated for administration via one or more of a number of routes including, but not limited to, buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutically acceptable carrier, diluent or excipient” generally does not provide any pharmacological activity to the formulation, though it may provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000.

A “pharmaceutically acceptable carrier, diluent or excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration.

Pharmaceutically acceptable carriers include sterile aqueous solutions or dispersions and sterile powders for the extemporaneous preparation of sterile injectable solutions or dispersions. The use of such media and agents for pharmaceutically active substances is well-known in the art. Except insofar as any conventional media or agent is incompatible with the active compound, use thereof in the pharmaceutical compositions disclosed herein is contemplated. Supplementary active compounds can also be incorporated into the compositions.

Pharmaceutical compositions can be, and desirably are, sterile and stable under the conditions of manufacture and storage. The composition can be formulated as a solution, microemulsion, liposome, or other ordered structure suitable for high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion, and by the use of surfactants.

In many cases, it can be desirable, and even preferable, to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds can be formulated in a time-release formulation, for example in a composition that includes a slow-release polymer. The active compounds can be prepared with carriers that will protect the compound against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated. The pharmaceutical composition can be orally administered as a capsule (hard or soft), tablet (film-coated, enteric-coated or uncoated), powder or granules (coated or uncoated) or liquid (solution or suspension). The formulations may be conveniently prepared by any of the methods well-known in the art. The pharmaceutical compositions can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically acceptable dosage form known to persons of ordinary skill in the art, and combinations thereof, are contemplated. Examples of such dosage forms include, without limitation, chewable tablets, quick-dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds, which can be included by admixture are, for example, medically inert ingredients (e.g., solid and liquid diluents), such as lactose, dextrosesaccharose, cellulose, starch or calcium phosphate for tablets or capsules, olive oil or ethyl oleate for soft capsules, and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate; binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of compound in a pharmaceutical composition may vary according to factors such as the disease state, age, gender, weight, patient history, risk factors, predisposition to disease, administration route, and pre-existing treatment regime (e.g., possible interactions with other medications) of the individual. Dosage regimens may be adjusted to provide the optimum inhibition of NTMT1. For example, a single bolus may be administered, several divided doses may be administered over time, or the dose may be proportionally reduced or increased as indicated by the exigencies of situation.

“Dosage unit form” refers to physically discrete units suited as unitary dosages for the subject (e.g., a mammalian subject); each unit containing a predetermined quantity of active compound calculated to produce the desired level of NTMT1 inhibition in association with the required pharmaceutical carrier. The specification for the dosage unit forms are dictated by, and directly dependent on, the unique characteristics of the active compound, and the limitations inherent in the art of compounding such an active compound for individuals with pre-existing sensitivities.

A dosage is typically administered once, twice, or thrice a day, although more frequent dosing intervals are possible. The dosage may be administered every day, every 2 days, every 3 days, every 4 days, every 5 days, every 6 days, and/or every 7 days (i.e., once a week). In one embodiment, the dosage may be administered daily for up to and including 30 days, preferably between 7-10 days. In another embodiment, the dosage may be administered twice a day for 10 days. If the patient has a chronic disease or condition for which the inhibition of NTMT1 is beneficial, the dosage may be administered for as long as signs and/or symptoms persist. The patient may require “maintenance treatment” where the patient is receiving dosages every day for months, years, or the remainder of their lives. In addition, the composition may be administered to effect prophylaxis of recurring symptoms or signs. For example, the dosage may be administered once or twice a day to prevent the onset of symptoms in a subject at risk, especially an asymptomatic subject.

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of a disease, e.g., inflammation, or systemic, wherein the composition is given to the subject and passes through the body widely, thereby reaching the site(s) of disease. Local administration can be administration to the cell, tissue, organ, and/or organ system, which encompasses and/or is affected by a disease, and/or where the disease signs and/or symptoms are active or are likely to occur. Administration can be topical with a local effect, i.e., the composition is applied directly where its action is desired. Administration can be enteral wherein the desired effect is systemic (non-local), i.e., the composition is given via the digestive tract. Administration can be parenteral, where the desired effect is systemic, i.e., the composition is given by other routes than the digestive tract.

Thus, in further view of the above, also provided is a method of inhibiting NTMT1 in a subject in need thereof. The method comprises administering an effective amount of (i) an above-described compound, which inhibits NTMT1, or (ii) a pharmaceutical composition comprising an above-described compound, which inhibits NTMT1, and a pharmaceutically acceptable carrier, diluent, excipient, or a combination thereof. The subject can have cancer, such as, but not limited to, cervical, prostate, lung, breast, colorectal, or pancreatic cancer, melanoma, or neuroblastoma.

In some other embodiments, the method comprises administering a composition comprising an effective amount of a compound, which inhibit NTMT1, together with a therapeutically effective amount of one or more other compounds with the same or different mode of action to a subject, such as a subject with cancer.

The term “effective amount” refers to that amount of one or more compounds that inhibits NTMT1 in a tissue system, animal or human, that is being sought by a researcher, veterinarian, medical doctor or other clinician. The inhibition of NTMT1 can include the alleviation of signs and symptoms of a disease, such as cancer (e.g., cervical, prostate, lung, breast, colorectal, or pancreatic cancer), melanoma or neuroblastoma. In some embodiments, the effective amount is that which may inhibit NTMT1, including the alleviation of signs and symptoms of a disease, such as cancer (e.g., cervical, prostate, lung, breast, colorectal, or pancreatic cancer), melanoma or neuroblastoma at a reasonable benefit/risk ratio applicable to any medical treatment. However, it is to be understood that the total daily usage of the compounds and compositions described herein may be decided by the attending physician within the scope of sound medical judgment. The specific effective dose level for any particular subject will depend upon a variety of factors, including a pre-existing disease or disorder and the severity thereof; the activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well-known to the researcher, veterinarian, medical doctor or other clinician. It is also appreciated that the effective amount can be selected with reference to any toxicity, or other undesirable side effect, that might occur during administration of one or more of the compounds.

In addition to the illustrative dosages and dosing protocols described herein, it is to be understood that an effective amount of any one or a mixture of the compounds can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of a pre-existing disease or disorder, the response of the individual subject, the particular compound administered, the mode of administration, the bioavailability characteristics of the preparation administered, the dose regimen selected, the use of concomitant medication, and other relevant circumstances.

The term “subject” includes human and non-human animals such as companion animals (dogs and cats and the like) and livestock animals. Livestock animals are animals raised for food production. The subject to be treated is preferably a mammal, in particular a human being.

ABBREVIATIONS: NTMT, protein N-terminal methyltransferase; SAM, S-5′-adenosyl-L-methionine; SAH, S-5′-adenosyl-L-homocysteines; SAHH, SAH hydrolase; MT, methyltransferase; PKMT, protein lysine methyltransferase; PRMT, protein arginine methyltransferase; PRMT1, protein arginine methyltransferase 1; TbPRMT7, Trypanosoma brucei protein arginine methyltransferase 7; G9a, euchromatic histone-lysine N-methyltransferase 2; SETD7, SET domain-containing protein 7; NNMT, nicotinamide N-methyltransferase; rt, room temperature; TFA, trifluoroacetic acid.

Protein N-terminal methyltransferase 1 (NTMT1) recognizes a unique N-terminal X-P-K/R motif (X represents any amino acid other than D/E) and transfer 1-3 methyl groups to the N-terminal region of its substrates. NTMT1 is implicated in cancer, such as colorectal cancer and malignant melanoma. Genetic studies indicate NTMT1 regulates mitosis and DNA damage repair. Guided by the co-crystal structures of NTMT1 in complex with the previously reported peptidomimetic inhibitor DC113, a series of new peptidomimetic inhibitors was designed and synthesized. Through a focused optimization of DC113, a new cell-potent peptidomimetic inhibitor was discovered. GD562 (IC₅₀=0.93±0.04 μM) exhibited improved inhibition on cellular N-terminal methylation levels of both the regulator of chromosome condensation 1 and oncoprotein SET, with an IC₅₀ value of ˜50 M in human colorectal cancer HCT116 cells. Notably, the inhibitory activity for the SET protein of GD562 increased over 6-fold compared to the previously reported cell-potent inhibitor DC541. Furthermore, GD562 also exhibited over 100-fold selectivity for NTMT1 against several other methyltransferases.

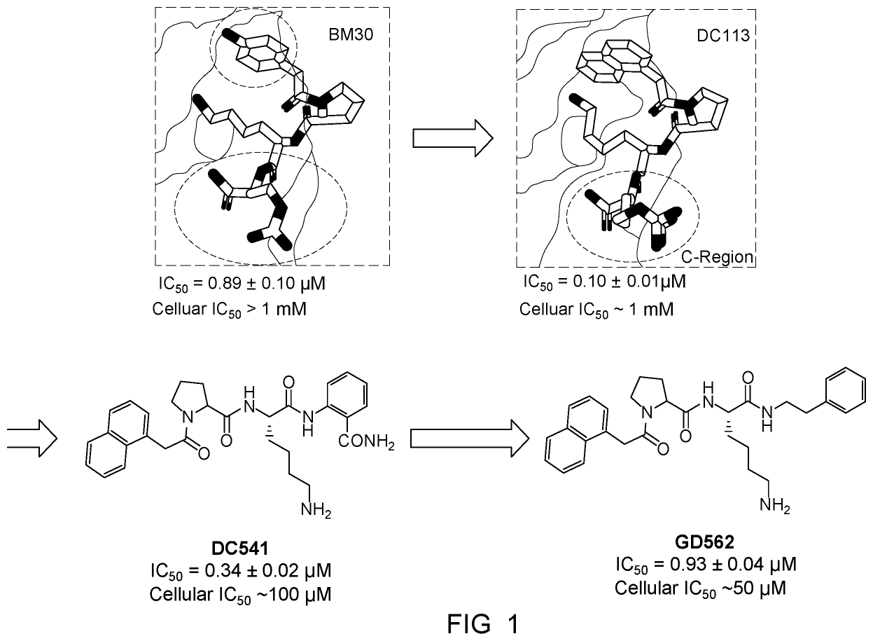

Guided by the unique substrate-binding mode of NTMT1/2, several peptidomimetic inhibitors with modest cellular potency have been reported.^{14, 15} The first peptidomimetic inhibitor BM30 displayed an IC₅₀ value of 0.89±0.1 μM and over 100-fold selectivity for NTMT1/2 among a panel of 41 methyltransferases.¹⁴ To improve the cellular uptake of peptidomimetic inhibitors,¹⁴ substituting the phenol group of BM30 with a naphthalene ring yielded DC113 with improved inhibition activity (IC₅₀=0.1±0.01 μM) and cell permeability (FIG. 1).¹⁵ However, DC113 only decreased ˜50% of the cellular me3-RCC1 level at 1 mM in HCT116 cells.¹⁵ The co-crystal structure of NTMT1-DC113 (FIG. 1) confirmed a similar binding mode of DC113 as the peptide substrate SPKRIA.¹⁵ As the Pro2 and Lys3 moieties of in the peptide substrates and peptidomimetic inhibitors are important to the binding,¹⁴ subsequent replacement of Arg4 of DC113 with benzamide provided DC541, enhancing cellular inhibition (IC₅₀ of ˜100 μM) on the me3-RCC1 level in HCT116 compared to DC113.¹⁵ Thus, it was hypothesized that further introduction of hydrophobic groups would promote the cellular potency without interfering with binding to NTMT1. Herein, focus was placed on optimization of the C-region of DC113 or DC541 to enhance cellular potency (FIG. 1).

### Design

Compared to DC113, DC541 exhibited about 10-fold enhanced cellular inhibition with a minor ˜3-fold decrease in its inhibitory activity on recombinant NTMT1, suggesting that optimization of the C-region of the ligand scaffold is a feasible strategy to improve the hydrophobicity and enhance the cell potency of the compounds (FIG. 1). As the C-region is exposed to solvent, minimal interferences to the interaction of the compound with NTMT1 were expected. In addition, incorporating non-amino acid functional groups has the potential to improve the compound's proteolytic stability.¹⁵ Therefore, it was hypothesized that incorporation of more hydrophobic groups at the C-region of DC541 would increase ligand hydrophobicity, permeability, and proteolytic stability without significantly affecting the binding to NTMT1.14, 15 Thus, different R groups were introduced to increase the hydrophobicity considering its positive correlation to permeability,¹⁶ using cLogP as the parameter to track hydrophobicity in the design.¹⁷

### Synthesis

All the peptidomimetic inhibitors were synthesized in the solution phase, as shown in Scheme 1.¹⁵ Compound 2 was synthesized through the standard amide coupling of HCl·Lys (Boc)-COMe with Naphthalene-Pro and the subsequent hydrolysis provided 3. The final peptidomimetic inhibitors 1a-k were prepared by the reactions of various amines with 3 and followed by the subsequent acidic treatment with 4 N HCl in 1,4-dioxane.

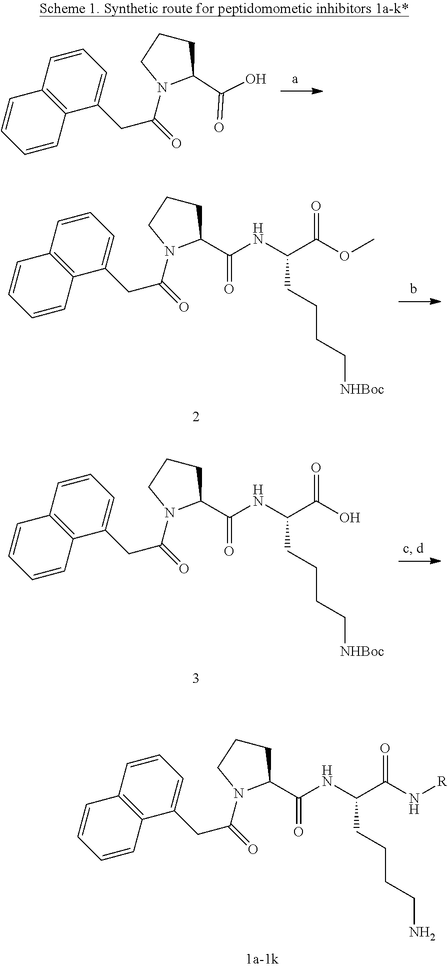

*Reagent and conditions: a) HCl·Lys (Boc)-COMe, HBTU, HOBt, DIPEA, DMF, r.t.; b) i. LiOH·H₂O, MeOH/H₂O, 0° C. to r.t.; c) R—NH₂, HBTU, HOBt, DIPEA, DMF, r.t.; d) 4N HCl in dioxane, 0° C. to r.t.

### Structure-Activity Relationship

Inhibitory activities of all synthesized compounds were determined with an established SAHH-coupled fluorescence assay.^{14, 15} To increase hydrophobicity, the benzamide ring was replaced with a bromophenyl group to produce 1a and 1b with higher cLogP values than DC541 (Table 1). Both compounds showed ˜2-fold increased inhibition compared to DC541, suggesting the bromide may form a halogen bond with NTMT1. Next, the carbon length between the phenyl ring and the amide group was varied to produce 1c-e, displaying inhibitory activities with IC₅₀ values ranging from 0.65 to 0.94 μM. The comparable inhibition of 1c-e suggested a marginal effect of chain linker length on the inhibitory activity. To explore the importance of aromaticity at the C-region, we replaced the phenyl ring of 1d with a cyclohexyl ring to produce 1g, exhibiting a comparable inhibition activity as 1d. As the introduction of the bromide at the phenyl ring led to ˜2-fold enhanced inhibition for 1a-b compared to DC541, we also added bromide to the phenyl ring of 1c at the meta- and para-positions to generate 1g and 1h, respectively. However, both 1g and 1h showed marginally improved potency (˜2-fold better) compared to 1c. As N-methylation has been proven to improve the cell permeability of peptides,¹⁸ a methyl group was introduced to the C-terminal amide of 1d to produce 1i but resulted in a 2-fold decreased inhibition. To explore possible interaction with Tyr215,^{15,19} a biphenyl group was synthesized at the C-terminal region to yield 1j (IC₅₀=2.5±0.09 UM), resulting in a ˜3-fold inhibition reduction compared to 1d. Then the substitution position of the phenyl ring was switched from the para to the meta-position to produce 1k (IC₅₀=0.69±0.08 μM), rescuing the inhibition with the comparable activity to the parent compound 1d.

TABLE 1
SAR of synthesized compounds with modifications at C-region

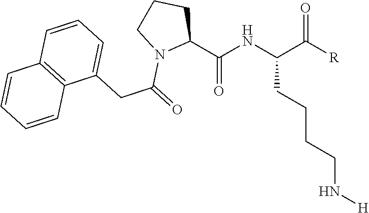

| | | | | IC₅₀/IC₅₀ |
|---|---|---|---|---|
| ID | R | IC₅₀ (μM) | cLogP | (DC541) |
| DC541 | 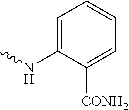 | 0.34 ± 0.03 | 0.7 | 1.0 |
| 1a (GD556) | 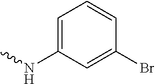 | 0.17 ± 0.008 | 2.9 | 0.50 |
| 1b (GD558) | 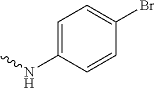 | 0.18 ± 0.007 | 2.9 | 0.53 |
| 1c (GD560) | 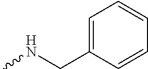 | 0.91 ± 0.03 | 1.7 | 2.7 |
| 1d (GD562) | 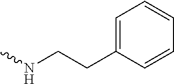 | 0.93 ± 0.04 | 1.9 | 2.7 |
| 1e (GD573) | 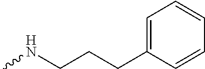 | 0.65 ± 0.01 | 2.3 | 1.9 |
| 1f (GD564) | 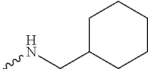 | 0.94 ± 0.03 | 2.4 | 2.8 |
| 1g (GD566) | 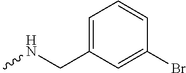 | 0.58 ± 0.03 | 2.5 | 1.7 |
| 1h (GD568) | 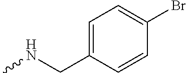 | 0.82 ± 0.07 | 2.5 | 2.4 |
| 1i (GD589) | 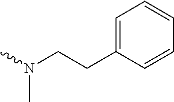 | 1.7 ± 0.07 | 2.3 | 5.0 |
| 1j (GD590) | 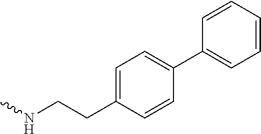 | 2.5 ± 0.09 | 3.8 | 7.4 |
| 1k (GD591) | 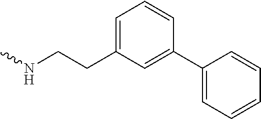 | 0.69 ± 0.08 | 3.8 | 2.0 |

*N.D. = not determined. All experiments were performed in duplicate (n = 2) and presented as mean ± standard deviation (SD).

### Computational Studies

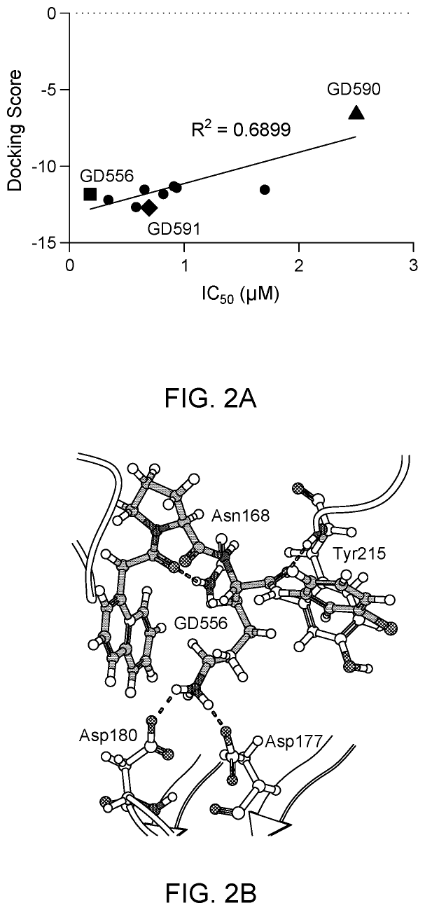

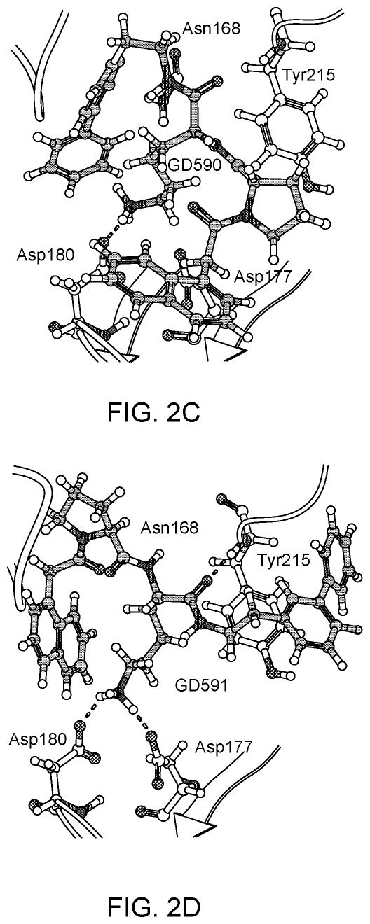

To understand the contribution of different functional groups to the inhibition activities of the peptidomimetic inhibitors, a docking study was performed using Glide software (Schrödinger).^{19, 20} The IC₅₀ values correlated well with the docking scores yielding an R² of ˜ 0.7 (FIG. 2A). All peptidomimetic inhibitors except 1j docked with a similar binding mode as DC541 and DC113. For example, the ε-NH₂ group of Lys3 side chain formed electrostatic interactions with Asp177 and Asp180, while the backbone carbonyl group interacted with Tyr215 through a hydrogen bond (FIG. 2B). In contrast, 1j only interacted with Asp180 and lost the hydrogen bonding interaction with Tyr215, providing a structural explanation behind its 8-fold decrease in inhibition activity compared to DC541 (FIG. 2C). In support of the hypothesis, the C-terminal biphenyl ring at the meta-position in compound 1k facilitated interactions with Asp 180, Asp 177, and Tyr215, with an additional π-π interaction between the fourth position phenyl ring and Tyr215 (FIG. 2D).

### Cytotoxicity

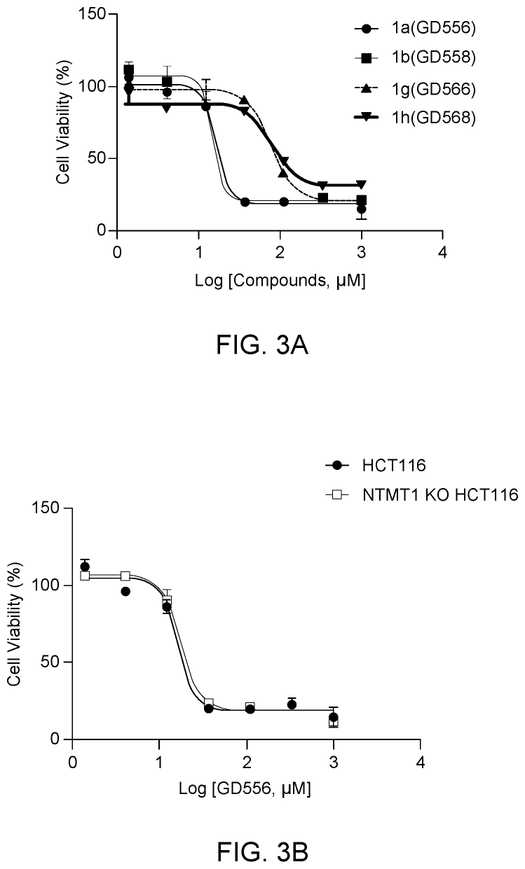

The cytotoxicity of the peptidomimetic inhibitors (IC₅₀<2 μM) was evaluated in HCT116 cells through alamaBlue assay as HCT116 cells are known for their high expression of NTMT1.15 Compared to DC541, all bromide compounds (1a, 1b, 1g, and 1h) and 1k displayed cytotoxicity with GI₅₀ values ranging from 48 μM to 135 μM (Table 2, FIG. 3A). Among them, 1a, 1b, and 1k exhibited GI₅₀ values of <70 μM, while 1g and 1h showed moderate growth inhibition against HCT116 with GI₅₀ values of ˜130 μM. All other compounds showed negligible inhibition of cell growth even at 300 μM. As NTMT1 knockout did not exhibit any significant inhibition on HCT116 cell growth, it was reasoned that inhibition of NTMT1 would not affect the HCT116 cell growth. Thus, it was hypothesized that the growth inhibition effects of these five peptidomimetic inhibitors may be caused by off-target effects. To test this hypothesis, compound 1a was selected to evaluate its effects on both wild-type and NTMT1 knockout HCT116 cells (FIG. 3B). Compound 1a exhibited a comparable GI₅₀ value on HCT116 cell growth regardless of the presence of NTMT1, suggesting the growth inhibition effect resulted from off-target effects.

### Inhibition on Cellular N-Terminal Methylation

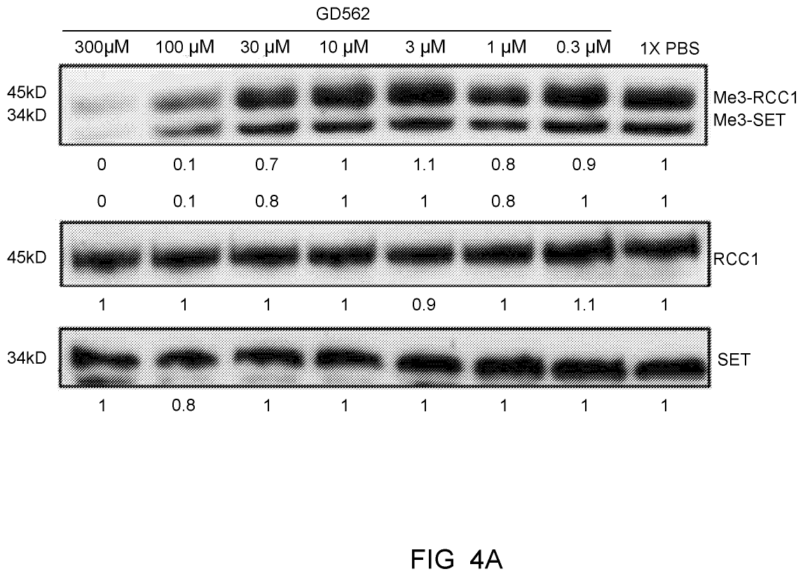

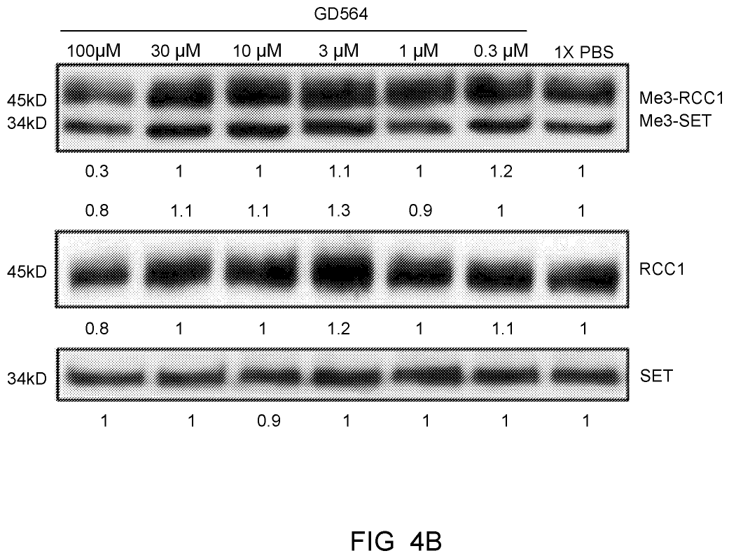

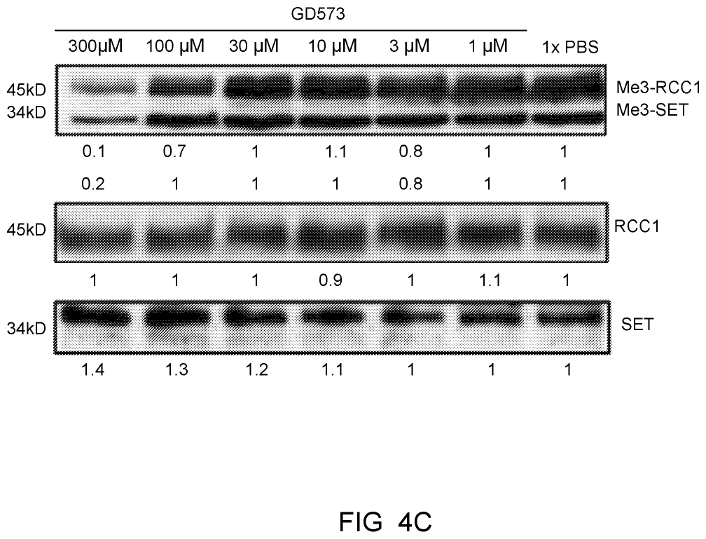

Next, the effects of all peptidomimetic inhibitors with GI₅₀>100 μM and IC₅₀<1 μM on the cellular α-N-terminal methylation level in HCT116 cells was examined.¹⁵ Compared to compound DC541,¹⁵ 1d-f displayed comparable or improved cellular inhibition activities on both me3-RCC1 and me3-SET (FIGS. 4A-4C). Among them, 1d (GD562) exhibited improved cellular inhibition on me3-RCC1 with an IC₅₀ of ˜50 μM (FIG. 4A). Furthermore, GD562 also inhibited me3-SET with a similar cellular IC₅₀ value of ˜50 μM, while DC541 did not show any inhibition of me3-SET level even at 300 μM in HCT116 cells.¹⁵ As biochemical IC₅₀ value of GD562 was about 3-fold higher than DC541, it was proposed that the improved cellular inhibition of GD562 may be attributed to its increased permeability with higher cLogP than DC541.

### Permeability

To understand the discrepancy between biochemical IC₅₀ and cellular IC₅₀ values, the ability of these inhibitors to cross the cell membrane in the Parallel Artificial Membrane Permeability Assay (PAMPA) was evaluated.²⁷⁻²⁹ The permeability of the control compound verapamil was determined as 8.6±0.98×10⁻⁶ cm/s, comparable to the literature value (8.8±0.53×10⁻⁶ cm/s).²⁹ As shown in Table 2, the majority of synthesized peptidomimetics showed 10- to 50-fold lower permeability compared to verapamil. The phenyl analogs 1c-1e with variable carbon length showed the lowest permeability values ranging from 0.17×10⁻⁶ cm/s to 0.26×10⁻⁶ cm/s which was about 2-fold less than the cyclohexyl analog 1f with a permeability value of 0.47×10⁻⁶ cm/s. This suggests that acyclic rings have better cell permeability than aromatic rings. Methylation of the C-terminal amide of 1d to 1i resulted in a higher cLogP, which was also evident in about 2-fold improvement in cell permeability due to the incorporation of a lipophilic methyl group. As expected, the biphenyl analogs 1j and 1k had the higher cell permeability values of 0.9×10⁻⁶ cm/s and 1.3×10⁻⁶ cm/s due to the improved lipophilicity of the additional phenyl group. This trend of the permeability values from the PAMPA assay is proportional to that of the cLogP values with 1j and 1k displaying the highest cLogP values of 3.8.

TABLE 2
Cellular studies of compounds 1c-h*

| | | | Cellular IC₅₀ | Permeability |
|---|---|---|---|---|
| ID | IC₅₀ (μM) | GI₅₀ (μM) | (μM) | (10⁻⁶ cm/s) |
| 1c (GD560) | 0.91 ± 0.03 | >300 | <300 | 0.24 ± 0.06 |
| 1d (GD562) | 0.93 ± 0.04 | >300 | ~50 | 0.26 ± 0.06 |
| 1e (GD573) | 0.65 ± 0.01 | >300 | ~100 | 0.17 ± 0.08 |
| 1f (GD564) | 0.94 ± 0.03 | >300 | ~100 | 0.47 ± 0.07 |
| 1g (GD566) | 0.58 ± 0.03 | 128 ± 20 | <300 | 0.6 ± 0.1 |
| 1h (GD568) | 0.82 ± 0.07 | 135 ± 23 | <300 | 0.9 ± 0.2 |
| 1j (GD590) | 2.5 ± 0.09 | N.D | N.D. | 0.9 ± 0.3 |
| 1k (GD591) | 0.69 ± 0.08 | 65 ± 7 | N.D. | ±0.4 |

*Note:
Verapamil was used as the positive control and was 8.6 ± 0.98 × 10⁻⁶ cm/s in the PAMPA assay. All experiments were performed in duplicates (n = 2) and presented as mean ± SD.
N.D. = not determined.

### Selectivity

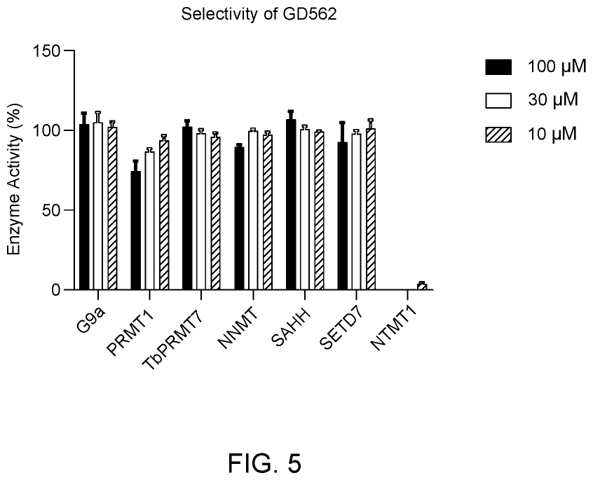

Since GD562 exhibited the highest cellular inhibition activity and lowest cytotoxicity among all tested peptidomimetic inhibitors (Table 2), the compound was chosen to perform in-house selectivity studies (FIG. 5) by examining its inhibition activity against a panel of five other SAM-dependent protein methyltransferases and the coupling enzyme SAHH in a fluorescence assay. The results showed that GD562 did not inhibit over 50% of those enzyme activities at 100 μM, indicating that the compound is more than 100-fold selective for NTMT1 against other methyltransferases and SAHH. Furthermore, compared to DC541, GD562 showed increased selectivity for NTMT1 over PRMT1.

### Discussion

In summary, a series of new peptidomimetic inhibitors was designed, synthesized, and evaluated based on the structures of previously reported inhibitors. The cellular inhibition activity of the peptidomimetic inhibitors was successfully optimized by introducing more hydrophobic function groups to replace the benzamide of DC541. The most cell-potent inhibitor GD562 exhibited over 100-fold selectivity over the other five SAM-dependent protein methyltransferases. Although GD562 showed decreased biochemical inhibition compared to DC541, it demonstrated an over 2-fold increase in cellular inhibition activity on me3-RCC1. In addition, it also inhibited the me3-SET with a similar IC₅₀ as me3-RCC1. Besides, GD562 exhibited improved selectivity than DC541 regarding PRMT1. Noticeably, the cellular inhibition activity of NTMT1 by GD562 is not optimal, since there is still a ˜50-fold difference between its enzymatic and cellular IC₅₀ values. Based on the PAMPA result, permeability alone did not explain the discrepancy between the biochemical and cellular IC₅₀ values. Future directions include further improving the cellular potency of these compounds by adding substituent groups to the C-terminal phenyl ring or testing new heterocycles at the C-region. Meanwhile, fluorescent labeling GD562 to monitor its cellular distribution would also be informative as NTMT1 is predominantly located in the nucleus.¹ Nevertheless, GD562 is the first tetra-peptidomimetic inhibitor that can inhibit both me3-RCC1 and me3-SET with an IC₅₀ value of ˜ 50 μM. Therefore, it can serve as a new lead compound for further optimization.

### Experimental Section

Protein expression and purification. Expression and purification of NTMT1, G9a, SETD7, PRMT1, NNMT, SAHH, and TbPRMT7 were performed as previously described. 30-35,14

NTMT1 biochemical assays. A fluorescence-based SAHH-coupled assay was applied to study the IC₅₀ values for all the compounds. The assay was performed under the following conditions in a final well volume of 40 μL: 5 mM Tris (pH=7.5), 50 mM KCl, 0.01% Triton X-100, 5 μM SAHH, 0.2 μM NTMT1, 100 μM SAM, and 15 μM ThioGlo1; or 25 mM Tris (pH 7.5), 50 mM KCl, 0.01% Triton X-100, 10 μM SAHH, 0.1 μM NTMT1, 100 μM SAM, and 10 μM ThioGlo4. The inhibitors were added at concentrations ranging from 0.15 nM to 10 μM. After 10 minutes of incubation, reactions were initiated by the addition of 0.5 μM GPKRIA peptide. Fluorescence was monitored on a BMG ClariOtar microplate reader with excitation 370 nm and emission 500 nm (for Thioglo 1), or excitation 400 nm and emission 465 nm (for Thioglo 4). Data were processed by using GraphPad Prism software 8.0.

Selectivity assays. The selectivity studies of G9a, SETD7, PRMT1, NNMT, and TbPRMT7 were performed through the SAHH-coupled fluorescence assay.³⁶ The inhibitor was added at three different concentrations (10, 30, 100 μM). The enzyme activity was normalized to the activity in the absence of the inhibitor. For G9a, the assay was performed in 1×PBS, pH7.4, 100 μM SAM, 10 μM SAH hydrolase, 25 nM G9a, DC562 (0, 10, 30, and 100 μM), 20 μM H3-15 peptide, and 15 μM Thioglo IV. For SETD7, the assay was performed in a final well volume of 40 μL: 25 mM potassium phosphate buffer (pH=7.6), 0.01% Triton X-100, 5 μM SAHH, 1 μM SETD7, 2 μM AdoMet, and 15 μM ThioGlo4. For PRMT1, the assay was carried out in 2.5 mM HEPES (pH 7.0), 25 mM NaCl, 25 μM EDTA, 50 μM TCEP, 100 μM SAM, 10 UM SAH hydrolase, 200 nM PRMT1, 100 μM H4-12 peptide, DC562 (0, 10, 30, and 100 μM), and 15 μM Thioglo4. For TbPRMT7, the assay was performed in a final well volume of 40 μL: 25 mM Tris (pH 7.5), 50 mM KCl, 0.01% Triton X-100, 5 μM SAHH, 0.2 μM TbPRMT7, 3 μM SAM, 60 μM H4-21 peptide, and 15 μM ThioGlo4. For NNMT, the assay was performed under the following conditions in a final well volume of 40 μL: 25 mM Tris (pH 7.5), 50 mM KCl, 0.01% Triton X-100, 5 μM SAHH, 0.1 μM NNMT, 10 UM nicotinamide, 10 μM SAM, and 10 μM ThioGlo4. Fluorescence intensity was monitored using a CLARIOstar microplate reader (Ex=370 nm, Em=500 nm) at 37° C. for 15 minutes. All experiments were determined in duplicate.

Cellular N-methylation level. HCT116 cells were seeded 40,000 cells/well on 24-well plates in the presence of 1×PBS or DC541 at different concentrations for three days. Then cells were lysed in 1×RIPA cell lysis buffer (25 mM Tris-HCl, pH 7.6, 150 mM NaCl, 1% NP-40, 1% sodium deoxycholate, 0.1% SDS, protease inhibitors) and incubated for 30 minutes on ice. The cell lysates were centrifuged at 15,000 rpm for 10 minutes, and the precipitates were removed. The concentration of total protein was quantified by bicinchoninic acid (BCA) protein assay kit (ThermoFisher, #23228). Equal amounts of total protein were mixed with 4× loading dye and loaded onto a 12.5% SDS-PAGE gel and separated. The gel was transferred to a polyvinylidene difluoride (PVDF) membrane using the BioRad Trans-Blot Turbo system. The membrane was then blocked for 1 hour in 5% milk TBST solution and washed with 1×TBST solution three times. The membrane was incubated with the anti-me3-SPK antibody at 4° C. for 12 hours, washed with 1×TBST solution three times, and then incubated with Rabbit IgG-HRP antibody (Cell Signaling, #7074S) for 1 hour at room temperature. The membrane was again washed with 1×TBST solution three times and detected using a Protein Simple FluorChem imaging system. Image quantification was done using ImageJ software (NIH). All bands were compared to the respective untreated control, which was set at 1.0.

Cell viability assay. HCT116 cells were seeded as 5,000 cells/well on 96-well plates in the presence of 1×PBS or DC541 at different concentrations for 24, 48, or 72 hours. Cell viability was assessed using 0.2 mg/ml resazurin solutions prepared from resazurin sodium salt (Acros Organics™, AC418900050) dissolved in sterile 1×PBS. Then, the cells were incubated with 10 μl resazurin solution (10% of cell culture volume) for four hours at 37° C. The fluorescence was measured using a CLARIOstar microplate reader (Ex=540 nm, Em=620 nm) at 37° C. Cell viability was calculated as 100%×(fluorescence of treated cells-fluorescence of background controls)/(fluorescence of 1×PBS controls-fluorescence of background controls).

Parallel Artificial Membrane Permeability Assay.^{28,48} The PAMPA assay was performed with Corning BioCoat pre-coated PAMPA plate system (Corning 353015). The stock solutions in 10 mM DMSO was diluted with PBS to a final concentration of 200 μM with 2% DMSO (v/v). Verapamil was added as a control. The pre-coated PAMPA plate system was warmed to room temperature, and the donor plate (bottom) was filled with 300 μL of the diluted sample solution while the acceptor plate (top) was filled with 200 μL of the buffer. The acceptor plate was slowly lowered into the receiver plate and the assembly was incubated at room temperature for 5 hours. The plate sandwich was separated and the concentration of the sample in both the donor and receiver plate was determined by UV spectrometry using CLARIOstar plate reader set at 254 and 280 nM. The permeability of each compound was calculated using the formula:

$${P}_{e} = \frac{- In [ 1 - {C}_{A} ( t ) / {C}_{equilibrium} ]}{a * ( 1 / {V}_{D} + 1 / {V}_{A} ) * t}$$

$${C}_{equilibrium} = [ {C}_{D} ( t ) * {V}_{D} + {C}_{A} ( t ) * {V}_{A} ] / ( A * ( {V}_{D} + {V}_{A} ) )$$

### Wherein:

C_{D} (t)=compound concentration in donor well at time t.
C_{A} (t)=compound concentration in acceptor well at time t.
V_{D}=donor well volume
V_{A}=acceptor well volume
A=filter area
t=incubation time

### MS Characterization of Compounds:

Compound 1a (GD556). MALDI-MS (positive) m/z: calcd for C₂₉H₃₃BrN₄O₃ [M+H]⁺ m/z 567.1716, found m/z 567.3305.
Compound 1b (GD558). MALDI-MS (positive) m/z: calcd for C₂₉H₃₃BrN₄O₃ [M+H]⁺ m/z 567.1716, found m/z 567.3351.
Compound 1c (GD560). MALDI-MS (positive) m/z: calcd for C₃₀H₃₆N₄O₃ [M+H]⁺ m/z 501.2821, found m/z 501.4417.
Compound 1d (GD562). MALDI-MS (positive) m/z: calcd for C₃₁H₃₈N₄O₃ [M+H]⁺ m/z 515.2944, found m/z 501.4635.
Compound 1e (GD573). MALDI-MS (positive) m/z: calcd for C₃₂H₄₀N₄O₃ [M+H]⁺ m/z 529.3100, found m/z 529.4910.
Compound 1f (GD564). MALDI-MS (positive) m/z: calcd for C₃₀H₄₂N₄O₃ [M+H]⁺ m/z 507.3257, found m/z 507.4806.
Compound 1g (GD566). MALDI-MS (positive) m/z: calcd for C₃₀H₃₅BrN₄O₃ [M+H]⁺ m/z 579.1893, found m/z 579.3701.
Compound 1h (GD568). MALDI-MS (positive) m/z: calcd for C₃₀H₃₅BrN₄O₃ [M+H]⁺ m/z 581.1872, found m/z 581.3701.
Compound 1i (GD589). MALDI-MS (positive) m/z: calcd for C₃₁H₃₈N₄O₃ [M+H]⁺ m/z 529.2944, found m/z 529.4852.
Compound 1j (GD590). MALDI-MS (positive) m/z: calcd for C₃₇H₄₂N₄O₃ [M+H]⁺ m/z 591.3257, found m/z 591.5327.
Compound 1k (GD591). MALDI-MS (positive) m/z: calcd for C₃₇H₄₂N₄O₃ [M+H]⁺ m/z 591.3257, found m/z 591.5327.

### REFERENCES

1 Schaner Tooley, C. E.; Petkowski, J. J.; Muratore-Schroeder, T. L.; Balsbaugh, J. L.; Shabanowitz, J.; Sabat, M.; Minor, W.; Hunt, D. F.; Macara, I. G. NRMT is an α-N-methyltransferase that methylates RCC1 and retinoblastoma protein. Nature 2010, 466, 1125-1128.
2. Petkowski, J. J.; Bonsignore, L. A.; Tooley, J. G.; Wilkey, D. W.; Merchant, M. L.; Macara, I. G.; Schaner Tooley, C. E. NRMT2 is an N-terminal monomethylase that primes for its homologue NRMT1. Biochem J 2013, 456, 453-62.
3. Sathyan, K. M.; Fachinetti, D.; Foltz, D. R. α-amino trimethylation of CENP-A by NRMT is required for full recruitment of the centromere. Nature Communications 2017, 8, 14678.
4. Cai, Q.; Fu, L.; Wang, Z.; Gan, N.; Dai, X.; Wang, Y. α-N-Methylation of Damaged DNA-Binding Protein 2 (DDB2) and Its Function in Nucleotide Excision Repair. Journal of Biological Chemistry 2014.
5. Dai, X.; Rulten, S. L.; You, C.; Caldecott, K. W.; Wang, Y. Identification and Functional Characterizations of N-Terminal α-N-Methylation and Phosphorylation of Serine 461 in Human Poly(ADP-ribose) Polymerase 3. Journal of Proteome Research 2015, 14, 2575-2582.
6. Jia, K.; Huang, G.; Wu, W.; Shrestha, R.; Wu, B.; Xiong, Y.; Li, P. In vivo methylation of OLA1 revealed by activity-based target profiling of NTMT1. Chem Sci 2019, 10, 8094-8099.
7. Bade, D.; Cai, Q.; Li, L.; Yu, K.; Dai, X.; Miao, W.; Wang, Y. Modulation of N-terminal methyltransferase 1 by an N (6)-methyladenosine-based epitranscriptomic mechanism. Biochem Biophys Res Commun 2021, 546, 54-58.
8. Huang, R. Chemical Biology of Protein N-Terminal Methyltransferases. Chembiochem 2019, 20, 976-984.
9. Grimsley, G. R.; Scholtz, J. M.; Pace, C. N. A summary of the measured pK values of the ionizable groups in folded proteins. Protein Sci 2009, 18, 247-51.
10. Chen, T.; Muratore, T. L.; Schaner-Tooley, C. E.; Shabanowitz, J.; Hunt, D. F.; Macara, I. G. N-terminal α-methylation of RCC1 is necessary for stable chromatin association and normal mitosis. Nature Cell Biology 2007, 9, 596.
11. Bonsignore, L. A.; Butler, J. S.; Klinge, C. M.; Schaner Tooley, C. E. Loss of the N-terminal methyltransferase NRMT1 increases sensitivity to DNA damage and promotes mammary oncogenesis. Oncotarget 2015, 6, 12248-12263.
12. Zhang, J.; Song, H.; Chen, C.; Chen, L.; Dai, Y.; Sun, P. H.; Zou, C.; Wang, X. Methyltransferase-like protein 11A promotes migration of cervical cancer cells via up-regulating ELK3. Pharmacol Res 2021, 172, 105814.
13. Catlin, J. P.; Marziali, L. N.; Rein, B.; Yan, Z.; Feltri, M. L.; Schaner Tooley, C. E. Age-related neurodegeneration and cognitive impairments of NRMT1 knockout mice are preceded by misregulation of RB and abnormal neural stem cell development. Cell Death Dis 2021, 12, 1014.
14. Mackie, B. D.; Chen, D.; Dong, G.; Dong, C.; Parker, H.; Schaner Tooley, C. E.; Noinaj, N.; Min, J.; Huang, R. Selective Peptidomimetic Inhibitors of NTMT1/2: Rational Design, Synthesis, Characterization, and Crystallographic Studies. J Med Chem 2020, 63, 9512-9522.
15. Chen, D.; Dong, G.; Deng, Y.; Noinaj, N.; Huang, R. Structure-based Discovery of Cell-Potent Peptidomimetic Inhibitors for Protein N-Terminal Methyltransferase 1. ACS Med Chem Lett 2021, 12, 485-493.
16. Zhang, L.; Torgerson, T. R.; Liu, X. Y.; Timmons, S.; Colosia, A. D.; Hawiger, J.; Tam, J. P. Preparation of functionally active cell-permeable peptides by single-step ligation of two peptide modules. Proc Natl Acad Sci USA 1998, 95, 9184-9.
17. Ben-Dror, S.; Bronshtein, I.; Wiehe, A.; Roder, B.; Senge, M. O.; Ehrenberg, B. On the correlation between hydrophobicity, liposome binding and cellular uptake of porphyrin sensitizers. Photochem Photobiol 2006, 82, 695-701.
18. Li, Y.; Li, W.; Xu, Z. Improvement on Permeability of Cyclic Peptide/Peptidomimetic: Backbone N-Methylation as A Useful Tool. Mar Drugs 2021, 19.
19. Friesner, R. A.; Banks, J. L.; Murphy, R. B.; Halgren, T. A.; Klicic, J. J.; Mainz, D. T.; Repasky, M. P.; Knoll, E. H.; Shelley, M.; Perry, J. K.; Shaw, D. E.; Francis, P.; Shenkin, P. S. Glide: a new approach for rapid, accurate docking and scoring. 1. Method and assessment of docking accuracy. J Med Chem 2004, 47, 1739-49.
20. Friesner, R. A.; Murphy, R. B.; Repasky, M. P.; Frye, L. L.; Greenwood, J. R.; Halgren, T. A.; Sanschagrin, P. C.; Mainz, D. T. Extra precision glide: docking and scoring incorporating a model of hydrophobic enclosure for protein-ligand complexes. J Med Chem 2006, 49, 6177-96.
21. Kong, X.; Brooks, C. L. III 2 Dynamics: A New Approach to Free Energy Calculations. J. Chem. Phys. 1996, 105, 2414-2423.
22. Knight, J. L.; Brooks, C. L. III Multisite 2 Dynamics for Simulated Structure-Activity Relationship Studies. J. Chem. Theory Comput. 2011, 7, 2728-2739.
23. Keränen, H.; Pérez-Benito, L.; Ciordia, M.; Delgado, F.; Steinbrecher, T. B.; Oehlrich, D.; van Vlijmen, H. W. T.; Trabanco, A. A.; Tresadern, G. Acylguanidine Beta Secretase 1 Inhibitors: A Combined Experimental and Free Energy Perturbation Study. J. Chem. Theory Comput. 2017, 13, 1439-1453.
24. Wheeler, S. E.; Houk, K. N. Substituent Effects in the Benzene Dimer are Due to Direct Interactions of the Substituents with the Unsubstituted Benzene. J. Am. Chem. Soc. 2008, 130, 10854-10855.
25. Jorgensen, W. L.; Severance, D. L. Aromatic-Aromatic Interactions: Free Energy Profiles for the Benzene Dimer in Water, Chloroform, and Liquid Benzene. J. Am. Chem. Soc. 1990, 112, 4768-4774.
26. Nishio, M. CH/x hydrogen bonds in crystals. Cryst. Eng. Comm. 2004, 6, 130-158.
27. Kansy, M.; Senner, F.; Gubernator, K. Parallel Artificial Membrane Permeation Assay in the Description of Passive Absorption Processes. J. Med. Chem. 1998, 41 (7), 1007-1010.
28. Avdeef, A.; Strafford, M.; Block, E.; Balogh, M. P.; Chambliss, W.; Khan, I. Drug Absorption in Vitro Model: Filter-Immobilized Artificial Membranes: 2. Studies of the Permeability Properties of Lactones in Piper Methysticum Forst. Eur. J. Pharm. Sci. 2001, 14 (4), 271-280. doi[dot]org/10.1016/S0928-0987 (01) 00191-9.
29. Chen, X.; Murawski, A.; Patel, K.; Crespi, C. L.; Balimane, P. V. A Novel Design of Artificial Membrane for Improving the PAMPA Model. Pharm. Res. 2008, 25 (7), 1511-1520. doi[dot]org/10.1007/s11095-007-9517-8.
30. Richardson, S. L.; Mao, Y.; Zhang, G.; Hanjra, P.; Peterson, D. L.; Huang, R. Kinetic mechanism of protein N-terminal methyltransferase 1. J. Biol. Chem. 2015, 290, 11601-11610.
31. Dong, C.; Mao, Y.; Tempel, W.; Qin, S.; Li, L.; Loppnau, P.; Huang, R.; Min, J. Structural basis for substrate recognition by the human N-terminal methyltransferase 1. Genes Dev. 2015, 29, 2343-2348.
32. Wu, H.; Min, J.; Lunin, V. V.; Antoshenko, T.; Dombrovski, L.; Zeng, H.; Allali-Hassani, A.; Campagna-Slater, V.; Vedadi, M.; Arrowsmith, C. H.; Plotnikov, A. N.; Schapira, M. Structural biology of human H3K9 methyltransferases. PLOS One 2010, 5, e8570.
33. Feng, Y.; Xie, N.; Jin, M.; Stahley, M. R.; Stivers, J. T.; Zheng, Y. G. A transient kinetic analysis of PRMT1 catalysis. Biochemistry 2011, 50, 7033-44.
34. Barsyte-Lovejoy, D.; Li, F.; Oudhoff, M. J.; Tatlock, J. H.; Dong, A.; Zeng, H.; Wu, H.; Freeman, S. A.; Schapira, M.; Senisterra, G. A.; Kuznetsova, E.; Marcellus, R.; Allali-Hassani, A.; Kennedy, S.; Lambert, J. P.; Couzens, A. L.; Aman, A.; Gingras, A. C.; Al-Awar, R.; Fish, P. V.; Gerstenberger, B. S.; Roberts, L.; Benn, C. L.; Grimley, R. L.; Braam, M. J.; Rossi, F. M.; Sudol, M.; Brown, P. J.; Bunnage, M. E.; Owen, D. R.; Zaph, C.; Vedadi, M.; Arrowsmith, C. H. (R)-PFI-2 is a potent and selective inhibitor of SETD7 methyltransferase activity in cells. Proc Natl Acad Sci USA 2014, 111, 12853-8.
35. Debler, E. W.; Jain, K.; Warmack, R. A.; Feng, Y.; Clarke, S. G.; Blobel, G.; Stavropoulos, P. A glutamate/aspartate switch controls product specificity in a protein arginine methyltransferase. Proc Natl Acad Sci USA 2016, 113, 2068-73.
36. Chen, D.; Dong, G.; Noinaj, N.; Huang, R. Discovery of bisubstrate inhibitors for protein N-terminal methyltransferase 1. J. Med. Chem. 2019, 62, 3773-3779.
37. Pettersen, E. F.; Goddard, T. D.; Huang, C. C.; Couch, G. S.; Greenblatt, D. M.; Meng, E. C.; Ferrin, T. E. UCSF Chimera—a visualization system for exploratory research and analysis. J. Comput. Chem. 2004, 25, 1605-1612.
38. Best, R. B.; Mittal, J.; Feig, M.; Mackerell, A. D. Jr. Inclusion of Many-Body Effects in the Additive CHARMM Protein CMAP Potential Results in Enhanced Cooperativity of α-Helix And β-Hairpin Formation. Biophys. J. 2012, 103, 1045-1051.
39. Best, R. B.; Zhu, X.; Shim, J.; Lopes, P. E. M.; Mittal, J.; Feig, M.; MacKerell, A. D. Jr. Optimization of the Additive CHARMM All-Atom Protein Force Field Targeting Improved Sampling of the Backbone ¢, w, and Side-Chain χ1 and χ2 Dihedral Angles. J. Chem. Theory Comput. 2012, 8, 3257-3273.
40. Vanommeslaeghe, K.; Hatcher, E.; Acharya, C.; Kundu, S.; Zhong, S.; Shim, J.; Darian, E.; Guvench, O.; Lopes, P.; Vorobyov, I.; Mackerell, A. D. Jr. CHARMM General Force Field: A Force Field for Drug-Like Molecules Compatible with the CHARMM All-Atom Additive Biological Force Fields. J. Comput. Chem. 2010, 31, 671-690.
41. Vanommeslaeghe, K.; Mackerell, A. D. Jr. Automation of the CHARMM General Force Field (CGenFF) I: Bond Perception and Atom Typing. J. Chem. Inf. Model. 2012, 52, 3144-3154.
42. Vanommeslaeghe, K.; Raman, E. P.; Mackerell, A. D. Jr. Automation of the CHARMM General Force Field (CGenFF) II: Assignment of Bonded Parameters and Partial Atomic Charges. J. Chem. Inf. Model. 2012, 52, 3155-3168.
43. Jorgensen, W. L.; Chandrasekhar, J.; Madura, J. D.; Impey, R. W.; Klein, M. L. Comparison of Simple Potential Functions for Simulating Liquid Water. J. Chem. Phys. 1983, 79, 926-935.
44. Jo, S.; Kim, T.; Iyer, V. G.; Im W. J. Comput. Chem. 2008, 29, 1859-1865.
45. Steinbach, P. J.; Brooks, B. R. New Spherical-Cutoff Methods for Long-Range Forces in Macromolecular Simulation. J. Comp. Chem. 1994, 15, 667-683.
46. Hayes, R. L.; Armacost, K. A.; Vilseck, J. Z.; Brooks, C. L. III Adaptive Landscape Flattening Accelerates Sampling of Alchemical Space in Multisite λ Dynamics. J. Phys. Chem. B 2017, 121, 3626-3635.
47. The PyMOL Molecular Graphics System, Version 1.8, Schrodinger LLC, New York, New York, United States.
48. Kansy, M.; Senner, F.; Gubernator, K. Parallel Artificial Membrane Permeation Assay in the Description of Passive Absorption Processes. J. Med. Chem. 1998, 41 (7), 1007-1010.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, or within 99% or more of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms.

The terms and expressions, which have been employed, are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

What is claimed is:

1. A compound of formula (I): 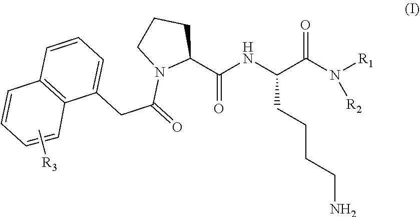
wherein:
R₁ is hydrogen or an alkyl;
R₂ is an alkyl, an alkenyl, an alkynyl, an arylalkyl, a cycloalkyl, a heterocyclyl, an aryl, a heteroaryl, an alkylaryl, or a heteroarylalkyl; and
R₃ represents seven substituents independently selected from hydrogen, an alkyl, an alkenyl, an alkynyl, an acyl, an amino, a cyano, a halo, hydroxy, a arylalkyl, a cycloalkyl, a heterocyclyl, an aryl, a heteroaryl, an alkylaryl, and a heteroarylalkyl;
or a pharmaceutically acceptable salt thereof.

2. The compound of claim 1, wherein (a) R₁ is optionally substituted, (b) R₂ is optionally substituted, (c) one or more of the R₃ substituents is/are substituted, or (d) two or more of (a)-(c).

3. The compound of claim 1, wherein R₁ is hydrogen, and R₂ is an arylalkyl.

4. The compound of claim 3, wherein the aryl of R₂ is substituted with a halo or an aryl.

5. The compound of claim 4, wherein the halo is Br.

6. The compound of claim 1, wherein R₁ is methyl, and R₂ is an arylalkyl.

7. The compound of claim 6, wherein the aryl of R₂ is substituted with a halo or an aryl.

8. The compound of claim 7, wherein the halo is Br.

9. The compound of claim 1, wherein R₁ is hydrogen, and R₂ is a cycloalkyl.

10. The compound of claim 1, wherein R₁ is methyl, and R₂ is a cycloalkyl.

11. The compound of claim 1, which is:

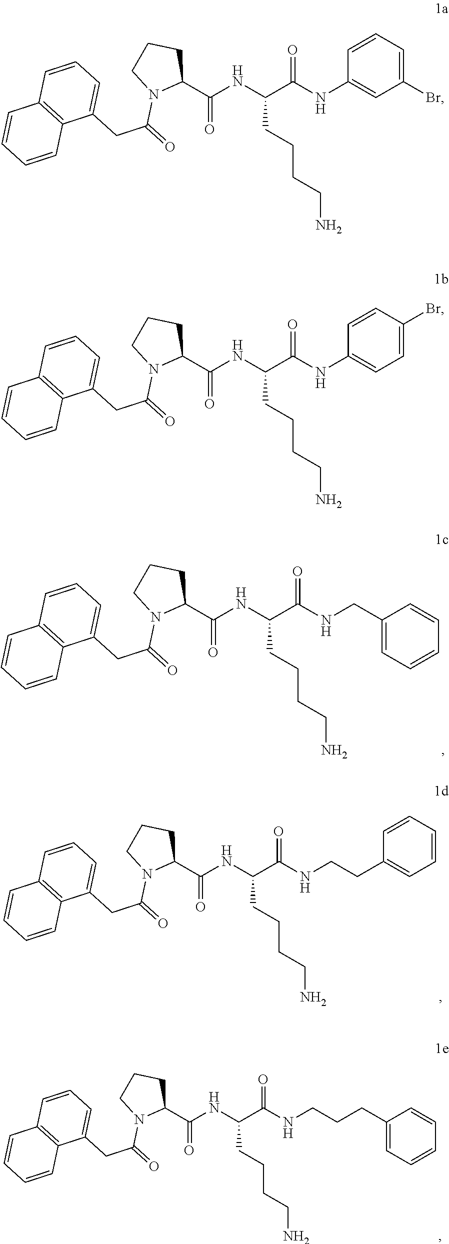 [SMILES: NCCCC[C@H](NC(=O)[C@@H]1CCCN1C(=O)Cc1cccc2ccccc12)C(=O)Nc1cccc(Br)c1 ; NCCCC[C@H](NC(=O)[C@@H]1CCCN1C(=O)Cc1cccc2ccccc12)C(=O)Nc1ccc(Br)cc1 ; NCCCC[C@H](NC(=O)[C@@H]1CCCN1C(=O)Cc1cccc2ccccc12)C(=O)NCc1ccccc1 ; NCCCC[C@H](NC(=O)[C@@H]1CCCN1C(=O)Cc1cccc2ccccc12)C(=O)NCCc1ccccc1 ; NCCCC[C@H](NC(=O)[C@@H]1CCCN1C(=O)Cc1cccc2ccccc12)C(=O)NCCCc1ccccc1]

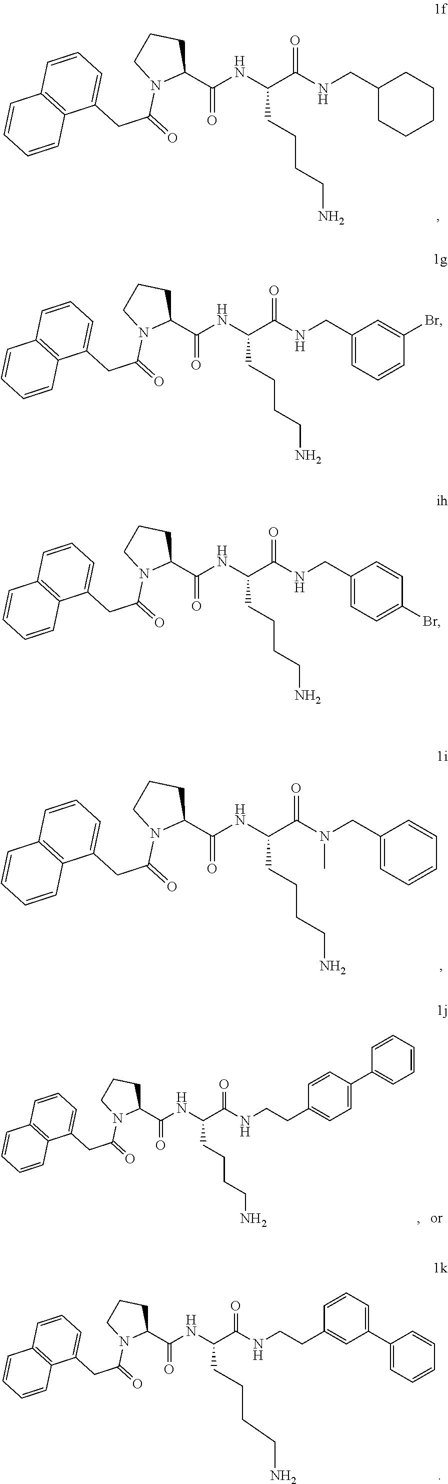 [SMILES: NCCCC[C@H](NC(=O)[C@@H]1CCCN1C(=O)Cc1cccc2ccccc12)C(=O)NCC1CCCCC1 ; NCCCC[C@H](NC(=O)[C@@H]1CCCN1C(=O)Cc1cccc2ccccc12)C(=O)NCc1cccc(Br)c1 ; NCCCC[C@H](NC(=O)[C@@H]1CCCN1C(=O)Cc1cccc2ccccc12)C(=O)NCc1ccc(Br)cc1 ; CN(Cc1ccccc1)C(=O)[C@H](CCCCN)NC(=O)[C@@H]1CCCN1C(=O)Cc1cccc2ccccc12 ; NCCCC[C@H](NC(=O)[C@@H]1CCCN1C(=O)Cc1cccc2ccccc12)C(=O)NCCc1ccc(-c2ccccc2)cc1 ; NCCCC[C@H](NC(=O)[C@@H]1CCCN1C(=O)Cc1cccc2ccccc12)C(=O)NCCc1cccc(-c2ccccc2)c1]

12. The compound of claim 1, which inhibits protein N-terminal methyltransferase 1.

13. A pharmaceutical composition comprising a compound of claim 1 and a pharmaceutically acceptable carrier, diluent, excipient or a combination thereof.

14. The pharmaceutical composition of claim 13, wherein the compound inhibits protein N-terminal methyltransferase 1.

15. A method of inhibiting N-terminal methyltransferase 1 (NTMT1) in a subject in need thereof, which method comprises administering an effective amount of (i) a compound of claim 12 or (ii) a pharmaceutical composition comprising the compound of claim 12 and a pharmaceutically acceptable carrier, diluent, excipient, or a combination thereof, whereupon NTMT1 in the subject is inhibited.

16. The method of claim 15, wherein the patient has cancer.

17. The method of claim 16, wherein the patient has cervical cancer, prostate cancer, lung cancer, breast cancer, colorectal cancer, pancreatic cancer, or melanoma.

18. The method of claim 16, wherein the patient has neuroblastoma.
